(19) United States
(12) Patent Application Publication
(10) Pub. No.: US 2024/0288262 A1
(43) Pub. Date: Aug. 29, 2024

(54) MANUAL CALIBRATION OF IMAGING SYSTEM

(71) Applicant: PHILIPS IMAGE GUIDED THERAPY CORPORATION, SAN DIEGO, CA (US)

(72) Inventors: Andreas JOHANSSON, SAN DIEGO, CA (US); Jason Y. SPROUL, WATERTOWN, MA (US)

(21) Appl. No.: 18/433,837

(22) Filed: Feb. 6, 2024

Related U.S. Application Data
(63) Continuation of application No. 17/194,729, filed on Mar. 8, 2021, now Pat. No. 11,892,289.
Application No. 17/194,729 is a continuation of application No. 14/107,861, filed on Dec. 16, 2013, now Pat. No. 10,942,022.
(60) Provisional application No. 61/739,881, filed on Dec. 20, 2012.

(51) Int. Cl. G01B 9/02091; A61B 5/00; G01B 9/02; G01B 9/02055; G01B 21/04; G06T 7/80

(52) U.S. Cl. CPC G01B 9/02091; A61B 5/0066; A61B 5/0073; G01B 9/02072; G01B 9/02089; G01B 21/045; G06T 7/80; A61B 5/7475; A61B 2560/0223; G06T 2207/10101; G06T 2207/20101; G06T 2207/30101

(57) ABSTRACT

The invention generally relates to methods for manually calibrating imaging systems such as optical coherence tomography systems. In certain aspects, an imaging system displays an image showing a target and a reference item. A user looks at the image and indicates a point within the image near the reference item. A processer detects an actual location of the reference item within an area around the indicated point. The processer can use an expected location of the reference item with the detected actual location to calculate a calibration value and provide a calibrated image. In this way, a user can identify the actual location of the reference point and a processing algorithm can give precision to the actual location.

### RELATED APPLICATION

The present application is a continuation of U.S. patent application Ser. No. 17/194,729, filed Mar. 8, 2021, now U.S. Pat. No. 11,892,289, which is a continuation of U.S. patent application Ser. No. 14/107,861, filed Dec. 16, 2013, now U.S. Pat. No. 10,942,022, which claims the benefit of and priority to U.S. Provisional Application No. 61/739,881, filed Dec. 20, 2012, each of which is hereby incorporated by reference in its entirety.

### TECHNICAL FIELD

The invention generally relates to methods for manually calibrating Time-of-Flight based imaging systems and interferometric systems more particularly, such as optical coherence tomography systems.

### BACKGROUND

Time-of-Flight imaging technologies in medicine and other fields involve measuring the time required for light to travel from a light sources to a target and back to a detector. Those measurements are used to provide high resolution images of the target. Time-of-Flight principles have applications in such diverse technologies as optical coherence tomography (OCT), gated viewing, positron emission tomography (PET), and radiotherapy. Beyond medical imaging, time-of-flight technologies are used in computer vision, robotics, art restoration, laser speed enforcement, and vision aids with security and military applications.

One problem that arises in many time-of-flight measurement technologies relates to calibration. Light that has been sent and received by an imaging component such as a lens or a catheter can be used to present an image of the target. But, where a reference point or zero point is not known a priori, the image does not necessarily contain calibration information relating to scale. Different approaches to calibrating these systems have included automatic computer processing algorithms as well as iterative user manipulation.

Known computer processing algorithms are limited. Typical approaches involve programming a computer to try to identify a reference point of a known dimension in the image. But where the known reference point appears among other images with similar shapes or is partially obscured and appears incompletely, computer processors are not adept at the induction required to determine the location or extent of the reference point.

Manual calibration is limited by the imprecision of human input and the time required for multiple iterations of spotting a calibration target and inputting information then zooming, centering, or focusing and repeating the steps. In, for example, the medical imaging context, the time involved is problematic because calibration often must occur while the patient is being examined. The imprecision is problematic for at least two reasons. First, the system must be calibrated precisely so that the imaging operation can be focused on the intended target (i.e., scanning at the desired depth in OCT). Also, tissue conditions such as tumors, plaque, or glaucoma must be measured precisely to monitor the progress of the condition.

### SUMMARY

The invention generally provides systems and methods for manually calibrating an imaging system in which a user looks at an image of a target and indicates a point near a location of a reference point within the image. An image processing operation is employed to determine the precise location of the reference point. Thus, a user can identify the actual location of the reference point and a processing algorithm can give precision to the actual location. Where the reference point is, for example, a physical feature that gets imaged while the target is imaged, information about the expected location of that physical feature may be independently provided to the system. The system calculates a calibration value based on the expected and actual locations and adjusts to display an image at a known scale. Where the imaging system is operating live, it can take new images, providing them at the known scale. Where a user is reviewing stored images, the imaging system can adjust those stored images to provide them at a known scale. Because images are provided at a known scale, imaging systems can be focused on the intended target and the resulting images reveal dimensions of target subject matter. For example, in medical imaging, the dimensions of a feature within tissue can be measured to monitor the progress of a condition.

Systems and methods of the invention have particular utility in interferometric imaging applications where light from a reference path is combined with light from a sample path and the resulting interference pattern is analyzed. In OCT, for example, an interferometer is used to split light into fiber optic-based sample and reference paths. The length of the reference path must be adjusted to match the length of the sample path as defined by the outer surface of the imaging catheter sheath. The difference between the length of the sample and the reference path is the z-offset, which is zero when the paths have matched lengths. If the z-offset is known, the system can be calibrated by changing the length of the reference path to match the length of the sample path. This can be accomplished, for example, by operating a motor within a variable delay line (VDL) in the reference path. The invention provides methods for calibrating an interferometric imaging system by determining a z-offset of the system and using the determined z-offset value to provide an image at a known scale.

In certain aspects, the invention provides a method of calibrating an imaging system by displaying an image showing a target and a reference item, receiving user input indicating a point within the image, and detecting a location of the reference item within an area around the indicated point. If the reference is not detected within the area, the area may be expanded and the detection step repeated. The detected location is used to calculate a calibration value and a calibrated image of the target at a known scale is provided.

In some embodiments, the imaging system is an optical coherence tomography system. The reference item can be an image of a catheter sheath (e.g., a known surface such as the outer surface of the sheath). A scan from the system can be displayed, for example, on a computer monitor in tomographic view or in an image-longitudinal display. A user of the system can identify the catheter sheath and indicate its location by an input gesture, such as clicking with a mouse or touching a touchscreen. The reference item can be detected by a morphological image processing operation such as, for example, erosion, dilation, or a combination thereof. Where the imaging system is an intravascular OCT system, the catheter sheath may appear generally as a vertical lineal element in a B-scan.

A processor can begin by analyzing, for example, an area of the B-scan around a point corresponding to the user's input. Thus the user input is taken as a starting point, and image processing is performed to identify the reference item (catheter sheath) within the area around the point. Using signal processing operations, the processing system finds a line in the area, for example, the highest valued contiguous line. The processing system can extrapolate and expand a search or processing algorithm. For example, where the line is substantially vertical, the system looks up and down to identify a location of substantially all of the catheter sheath.

In some OCT operations, an imaging catheter is associated with a specific sample path length. Path length may be provided with each catheter, for example, by a manufacturer. The catheter sample path length can give an expected location of the reference point. Where the expected location is thus provided, a difference between the actual location and the expected location can be used to detect and correct for, for example, path length changes (e.g., stretching) during operation.

With a calibration value calculated, the imaging system can provide a calibrated image—either in live mode, by making a new scan, or in review mode, by transforming stored image data.

In related aspects, the invention provides an imaging system that includes a processor and a computer-readable storage medium having instructions therein which can be executed to cause the system to display an image showing a target and a reference item, receive user input indicating a point within the image, and detect a location of the reference item within an area around the indicated point. The system uses the detected location to calculate a calibration value and provide a calibrated image of the target at a known scale.

In other aspects, the invention provides a method of calibrating an imaging system by displaying an image showing a target and a reference item, receiving user input indicating a motion of the reference item within the image, and calculating a calibration value based on indicated motion of the reference item. For example, a user can use a mouse to drag an image of the reference item onto a calibration mark, as seen on a computer screen. The user input indicating a motion of the reference item can be a drag-and-drop operation performed with a computer pointing device (e.g., mouse or trackpad), a drag along a touchscreen, or any other suitable computer input method. The motion indicated by the input is used to calculate the calibration value. Based on the calculated calibration value, a scaled image of the target is provided.

Methods of the invention include transforming the reference item within the image by, for example, re-sizing, rotation, translating, or a combination thereof. In some embodiments, the system is an interferometric imaging system and the reference item is a portion of the system itself. For example, where the reference item is an image of an OCT catheter sheath, the dragging motion can indicate a z-offset calibration value, i.e., a change in a radius associated with a zero-point in the image. The z-offset calibration can be accomplished by moving a VDL motor or transforming image data.

In some embodiments, the user input is received, and then the calibration operation (e.g., moving the VDL or transforming an existing image) is performed. In certain embodiments, the calibration operation is performed while the user input is received. Thus the user experiences that they are changing the image. Where an OCT system is used, the user experiences dragging the catheter sheath inwards or outwards (for example, to a reference calibration mark) and thus changing the image.

In some related aspects, the invention provides an imaging system that includes a processor and a computer-readable storage medium having instructions therein which can be executed to cause the system to display an image showing a target and a reference item, receive user input indicating a motion of the reference item within the image, and calculate a calibration value based on indicated motion of the reference item. The calibration value is used to provide a scaled image of the target.

### BRIEF DESCRIPTION OF THE DRAWINGS

FIG. 1 shows use of an imaging system according to certain embodiments.

FIG. 2 is a diagram of components of an OCT system.

FIG. 3 diagrams components within a patient interface module (PIM).

FIG. 4 shows the structure of a PIM according to certain embodiments.

FIG. 5 is a diagram of components in an imaging engine.

FIG. 6 is a diagram of an interferometer for use with systems of certain embodiments.

FIGS. 7A and 7B illustrate a segment of a blood vessel.

FIG. 8 shows the motion of parts of an imaging catheter according to certain embodiments of the invention.

FIG. 9 shows an array of A scan lines of a three-dimensional imaging system according to certain embodiments of the invention.

FIG. 10 shows the positioning of A scans with in a vessel.

FIG. 11 shows a B-scan.

FIG. 12 shows a tomographic view based on the B-scan of FIG. 10.

FIG. 13 illustrates a set of A scans used to compose a tomographic view.

FIG. 14 shows the set of A scans shown in FIG. 13 within a cross section of a vessel.

FIG. 15 shows a longitudinal plane through a vessel including several A scans.

FIG. 16 is a perspective view of an image longitudinal display (ILD) in the same perspective as the longitudinal plane shown in FIG. 15.

FIG. 17 shows a display of a system of the invention.

FIG. 18 is a display providing an image of the vessel shown in FIGS. 7A and 7B.

FIG. 19 illustrates receiving user input indicating a point within an image.

FIG. 20 shows an area around a point to be searched.

FIG. 21 shows a calibrated B-scan.

FIGS. 22 and 23 illustrates receiving user input indicating a motion.

FIG. 24 illustrates providing a scaled image based on an indicated motion.

FIG. 25 illustrates components of a system according to certain embodiments of the invention.

### DETAILED DESCRIPTION

The invention provides systems and methods for calibrating an imaging system. Systems and methods of the invention have application in imaging systems that require calibration to provide a scale. Exemplary systems include imaging and sensing systems based on principles of time-of-flight or coherent interference. In some embodiments, systems and applications contemplated for use with the invention include optical coherence tomography (OCT), time-of-flight cameras such as the CamCube 3.0 TOF camera sold under the trademark PDM[VISION] by PMDTechnologies GmbH (Siegen, Germany), or time-of-flight positron emission tomography (PET) technologies. See, e.g., Placht et al., 2012, Fast time-of-flight camera based surface registration for radiotherapy patient positioning, Med Phys 39:4-17; Karp et al., 2009, The benefit of time-of-flight in PET imaging, J Nucl Med 49:462-470. Other imaging systems for use with the invention include, for example, gated viewing, radiotherapy, intra-vascular ultrasound, magnetic resonance imaging, elastographic techniques such as magnetic resonance elastography or transient elastography systems such as FibroScan by Echosens (Paris, France), and electrical impedance tomography, as well as other applications in computer vision, robotics, art restoration, laser speed enforcement, and vision aids with security and military applications.

In OCT systems, a light source is used to provide a beam of coherent light. The light source can include an optical gain medium (e.g., laser or optical amplifier) to produce coherent light by stimulated emission. In some embodiments, the gain medium is provided by a semiconductor optical amplifier. A light source may further include other components, such as a tunable filter that allows a user to select a wavelength of light to be amplified. Wavelengths commonly used in medical applications include near-infrared light, for example between about 800 nm and about 1700 nm.

Generally, there are two types of OCT systems, common beam path systems and differential beam path systems, that differ from each other based upon the optical layout of the systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are further described for example in U.S. Pat. Nos. 7,999,938; 7,995,210; and 7,787,127 the contents of each of which are incorporated by reference herein in their entirety.

In a differential beam path system, the coherent light from the light source is input into an interferometer and split into a reference path and a sample path. The sample path is directed to the target and used to image the target. Reflections from the sample path are joined with the reference path and the combination of the reference-path light and the sample-path light produces interference patterns in the resulting light. The light, and thus the patterns, are converted to electric signals, which are then analyzed to produce depth-resolved images of the target tissue on a micron scale. Exemplary differential beam path interferometers are Mach-Zehnder interferometers and Michelson interferometers. Differential beam path interferometers are further described for example in U.S. Pat. Nos. 7,783,337; 6,134,003; and 6,421,164, the contents of each of which are incorporated by reference herein in its entirety.

Commercially available OCT systems are employed in diverse applications, including art conservation and diagnostic medicine, notably in ophthalmology where OCT can be used to obtain detailed images from within the retina. The detailed images of the retina allow one to identify diseases and trauma of the eye. Other applications of imaging systems of the invention include, for example, dermatology (e.g., to image subsurface structural and blood flow formations), dentistry (to image teeth and gum line), gastroenterology (e.g., to image the gastrointestinal tract to detect polyps and inflammation), and cancer diagnostics (for example, to discriminate between malignant and normal tissue).

In certain embodiments, systems and methods of the invention image within a lumen of tissue. Various lumen of biological structures may be imaged including, for example, blood vessels, including, but not limited, to vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, vagina, uterus and fallopian tubes, structures of the urinary tract including urinary collecting ducts, renal tubules, ureter, and bladder, and structures of the head and neck and pulmonary system including sinuses, parotid, trachea, bronchi, and lungs. Systems and methods of the invention have particular applicability in imaging veins and arteries such as, for example, the arteries of the heart. Since an OCT system can be calibrated to provide scale information, intravascular OCT imaging of the coronary arteries can reveal plaque build-up over time, change in dimensions of features, and progress of thrombotic elements. The accumulation of plaque within the artery wall over decades is the setup for vulnerable plaque which, in turn, leads to heart attack and stenosis (narrowing) of the artery. OCT images, if scaled or calibrated, are useful in determining both plaque volume within the wall of the artery and/or the degree of stenosis of the artery lumen. Intravascular OCT can also be used to assess the effects of treatments of stenosis such as with hydraulic angioplasty expansion of the artery, with or without stents, and the results of medical therapy over time.

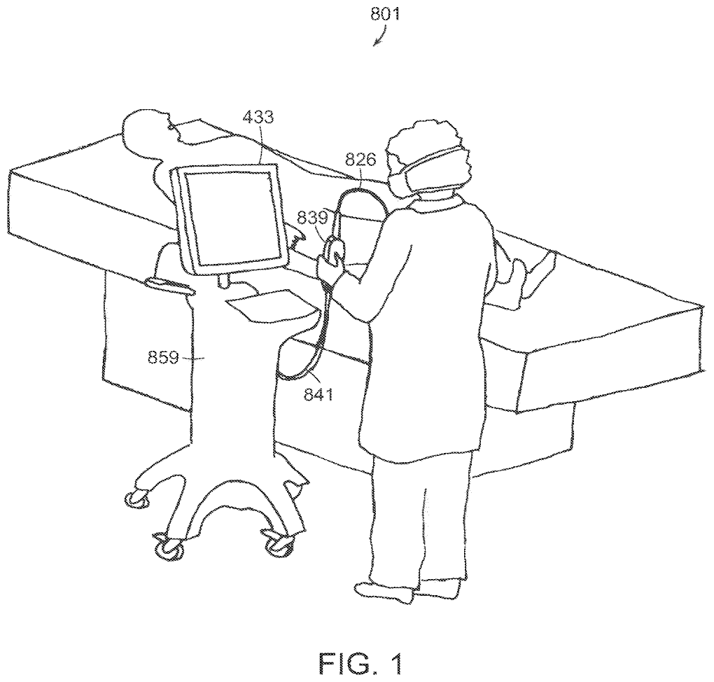

FIG. 1 depicts the use of an exemplary intravascular OCT system 801. A physician controls an imaging catheter 826 through use of a handheld patient interface module (PIM) 839 to collect image data from a patient. Image data collected through catheter 826 is transmitted by PIM cable 841 to an imaging engine 859, which can be, for example, housed within a bedside unit or in a nearby computer installation or in a server rack coupled via networking technologies. As shown in FIG. 1, an OCT system can further include a workstation 433 (e.g., a monitor, keyboard, and mouse).

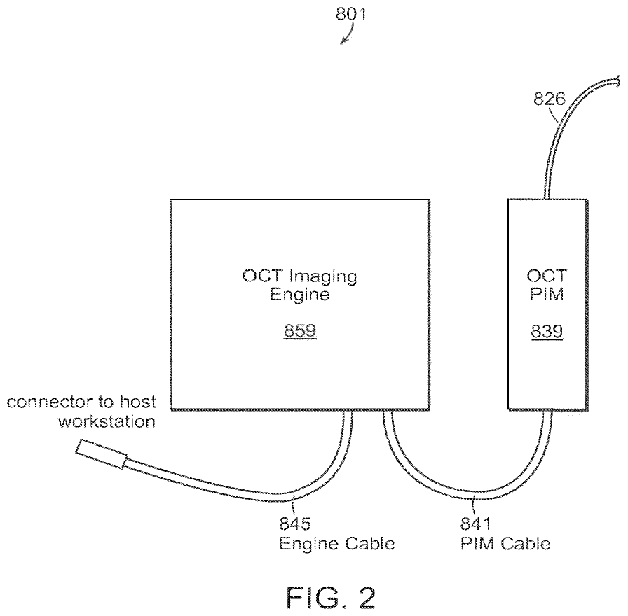

FIG. 2 gives a block diagram of components of OCT system 801. Imaging engine 859 is coupled to PIM 839 via PIM cable 841. Imaging catheter 826 extends from PIM 839 to the site of imaging. Engine cable 845 connects imaging engine 859 to host workstation 433. OCT is discussed in U.S. Pat. No. 8,108,030; U.S. Pub. 2011/0152771; U.S. Pub. 2010/0220334; U.S. Pub. 2009/0043191; U.S. Pub. 2008/0291463; and U.S. Pub. 2008/0180683, the contents of each of which are incorporated by reference in their entirety for all purposes. In certain embodiments, systems and methods of the invention include processing hardware configured to interact with more than one different three dimensional imaging system so that the tissue imaging devices and methods described here in can be alternatively used with OCT, IVUS, or other hardware.

As shown in FIG. 1, an operator controls imaging catheter 826 via handheld PIM 839. PIM 839 may include controls such as knobs or buttons to start or stop operation, set or vary speed or displacement, or otherwise control the imaging operation. PIM 839 further includes hardware for operating the imaging catheter.

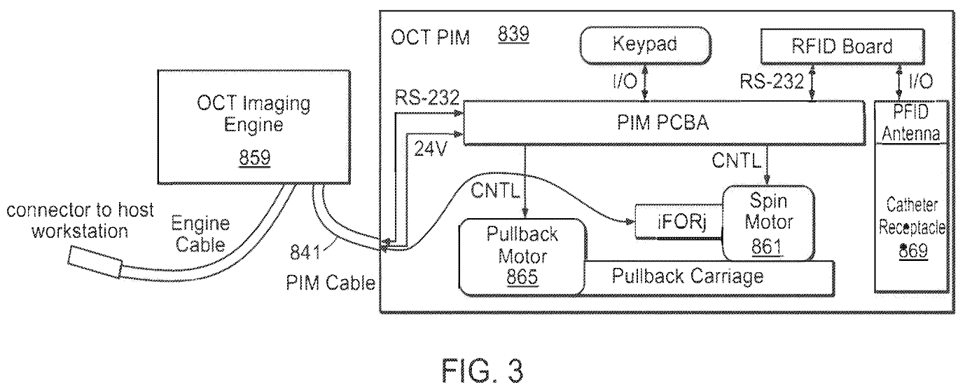

FIG. 3 shows components of PIM 839. Catheter 826 is mounted to PIM 839 via a catheter receptacle 869. Spin motor 861 is provided to rotate catheter 826 and pullback motor 865 is provided to drive lateral translation of catheter 826. Also depicted is a keypad for input/output, a fiber-optic rotary joint (iFORj), a printed circuit board assembly (PCBA), and optional RFID components.

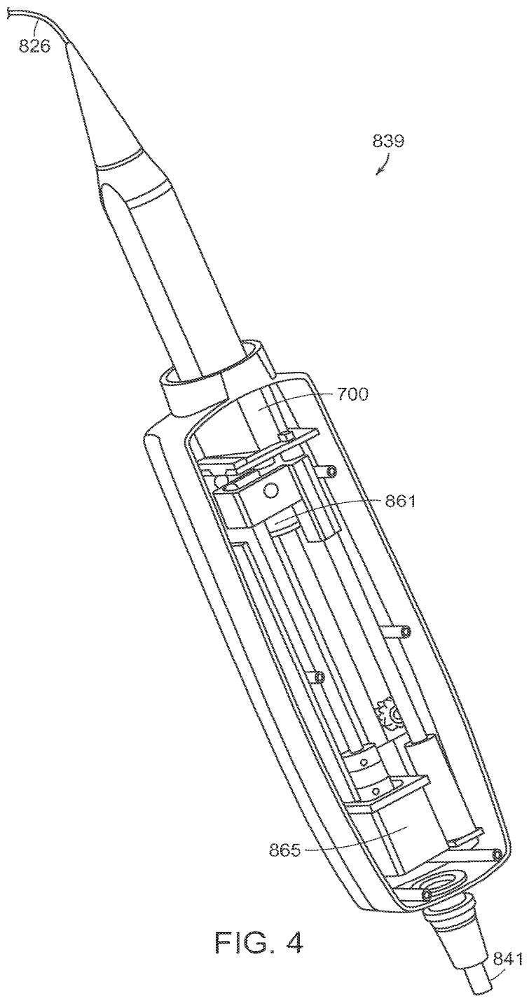

FIG. 4 gives a perspective view of PIM 839 with a keypad cover removed. Spin motor 861 is provided to rotate catheter 826 and pullback motor 865 causes lateral translation. Optical signals, electrical signals, or both arrive at PIM 839 via PIM cable 841. PIM cable 841 extends to imaging engine 859 as shown in FIG. 2.

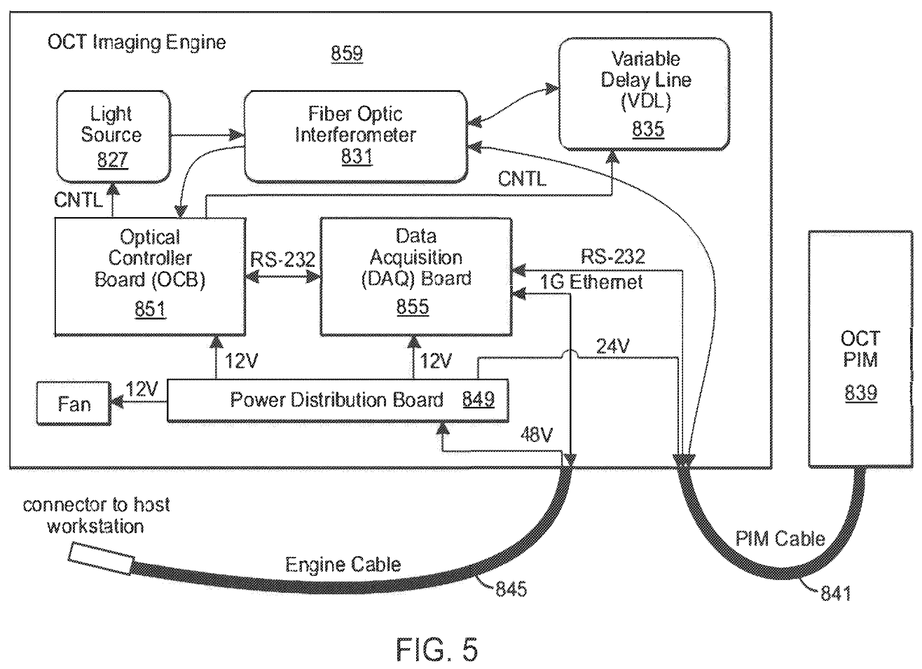

FIG. 5 shows components of imaging engine 859. As shown in FIG. 5, the imaging engine 859 (e.g., a bedside unit) houses a power distribution board 849, light source 827, interferometer 831, and variable delay line 835 as well as a data acquisition (DAQ) board 855 and optical controller board (OCB) 851.

Light source 827, as discussed above, may use a laser or an optical amplifier as a source of coherent light. Coherent light is transmitted to interferometer 831.

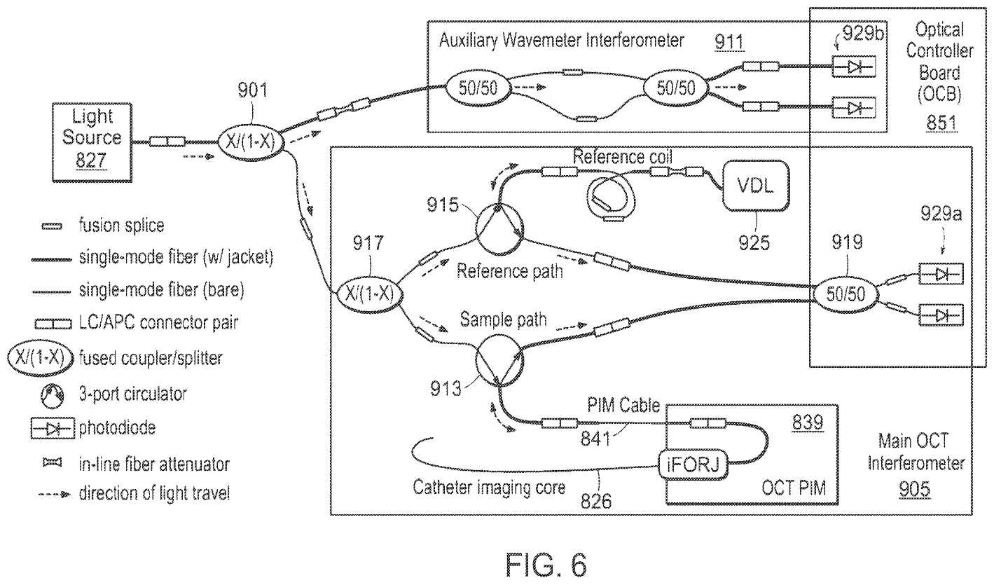

FIG. 6 shows a path of light through interferometer 831 during OCT imaging. Coherent light for image capture originates within the light source 827. This light is split between an OCT interferometer 905 and an auxiliary, or “clock”, interferometer 911. Light directed to the OCT interferometer is further split by splitter 917 and recombined by splitter 919 with an asymmetric split ratio. The majority of the light is guided into the sample path 913 and the remainder into a reference path 915. The sample path includes optical fibers running through the PIM 839 and the imaging catheter 826 and terminating at the distal end of the imaging catheter where the image is captured.

An image is captured by introducing imaging catheter 826 into a target within a patient, such as a lumen of a blood vessel. This can be accomplished by using standard interventional techniques and tools such as a guide wire, guide catheter, or angiography system. Suitable imaging catheters and their use are discussed in U.S. Pat. Nos. 8,116,605 and 7,711,413, the contents of which are incorporated by reference in their entirety for all purposes.

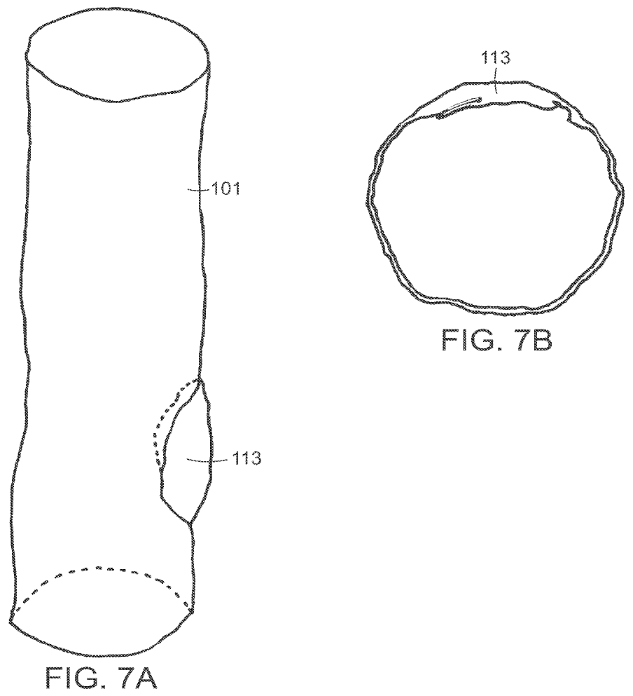

FIG. 7A provides an illustration of a segment of a vessel 101 having a feature 113 of interest. FIG. 7B shows a cross-section of vessel 101 through feature 113. In certain embodiments, intravascular imaging involves positioning imaging catheter 826 within vessel 101 near feature 113 and collecting data to provide a three-dimensional image. Data can be collected in three dimensions by rotating catheter 826 around a catheter axis to collect image data in radial directions around the catheter while also translating catheter 826 along the catheter axis. As a result of combined rotation and translation, catheter 826 collects image data from a series of scan lines (each referred to as an A-scan line, or A-scan) disposed in a helical array.

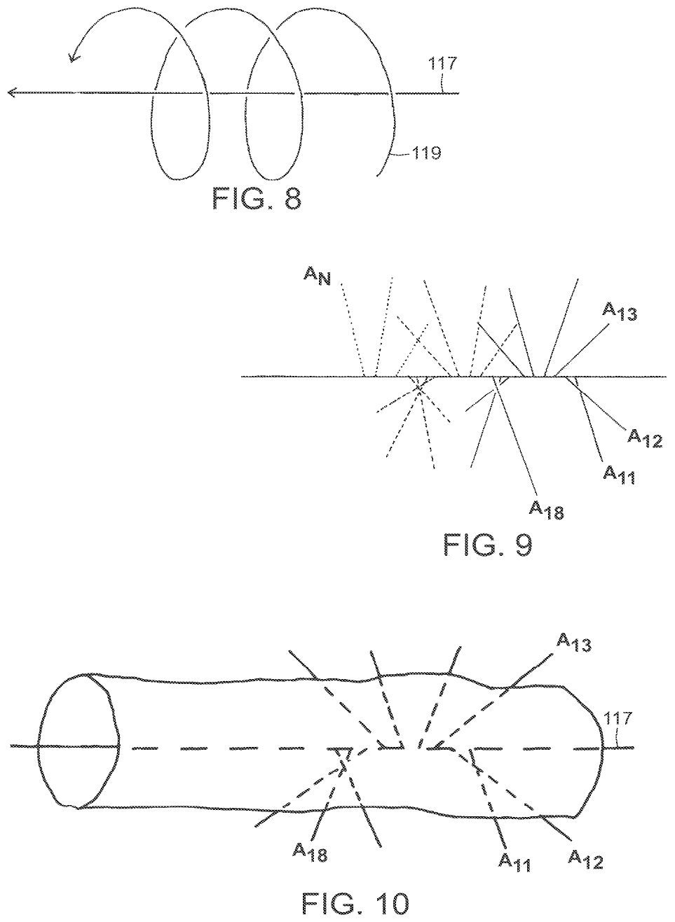

FIG. 8 shows the motion of parts of an imaging catheter according to certain embodiments of the invention. Rotation of imaging catheter 826 around axis 117 is driven by spin motor 861 while translation along axis 117 is driven by pullback motor 865, as discussed above with reference to FIG. 4. An imaging tip of catheter 826 generally follows helical trace 119, resulting in a motion for image capture described by FIG. 8. Blood in the vessel is temporarily flushed with a clear solution for imaging. When operation is triggered from PIM 839 or a control console, the imaging core of catheter 826 rotates while collecting image data, which data is delivered to the imaging system.

FIG. 9 illustrates the helical array of A-scan lines A₁₁, A₁₂, . . . , A_{N} captured by the imaging operation.

FIG. 10 is provided to show the positioning of A-scans A₁₁, A₁₂, . . . , A_{N} within vessel 101. Each place where one of A-scans A₁₁, A₁₂, . . . , A_{N} intersects a surface of a feature within vessel 101 (e.g., a vessel wall) coherent light is reflected and detected. Catheter 826 translates along axis 117 being pushed or pulled by pullback motor 865.

Looking back at FIG. 6, the reflected, detected light is transmitted along sample path 913 to be recombined with the light from reference path 915 at splitter 919. Calibration of the system relates to a length of sample path 913 compared to a length of reference path 915. The difference between these lengths is referred to as the z-offset and when the paths are the same length, the z-offset is said to be zero, and the system is calibrated. Calibration will be discussed in more detail below. Z-offset is discussed in U.S. Pat. No. 8,116,605, the contents of which are hereby incorporated by reference in their entirety for all purposes. When the z-offset is zero, the system is said to be calibrated.

After combining light from the sample, and reference paths, the combined light from splitter 919 is split into orthogonal polarization states, resulting in RF-band polarization-diverse temporal interference fringe signals. The interference fringe signals are converted to photocurrents using PIN photodiodes 929a, 929b, . . . on the OCB 851 as shown in FIG. 6. The interfering, polarization splitting, and detection steps are done by a polarization diversity module (PDM) on the OCB. Signal from the OCB is sent to the DAQ 855, shown in FIG. 5. The DAQ includes a digital signal processing (DSP) microprocessor and a field programmable gate array (FPGA) to digitize signals and communicate with the host workstation and the PIM. The FPGA converts raw optical interference signals into meaningful OCT images. The DAQ also compresses data as necessary to reduce image transfer bandwidth to 1 gigabit per second (Gbps) (e.g., compressing frames with a lossy compression JPEG encoder).

Data is collected from A-scans A₁₁, A₁₂, . . . , A_{N}, as shown in FIG. 10, and stored in a tangible, non-transitory memory. A set of A-scans captured in a helical pattern during a rotation and pullback event can be collected and viewed alongside one another in a plane, in a format known as a B-scan.

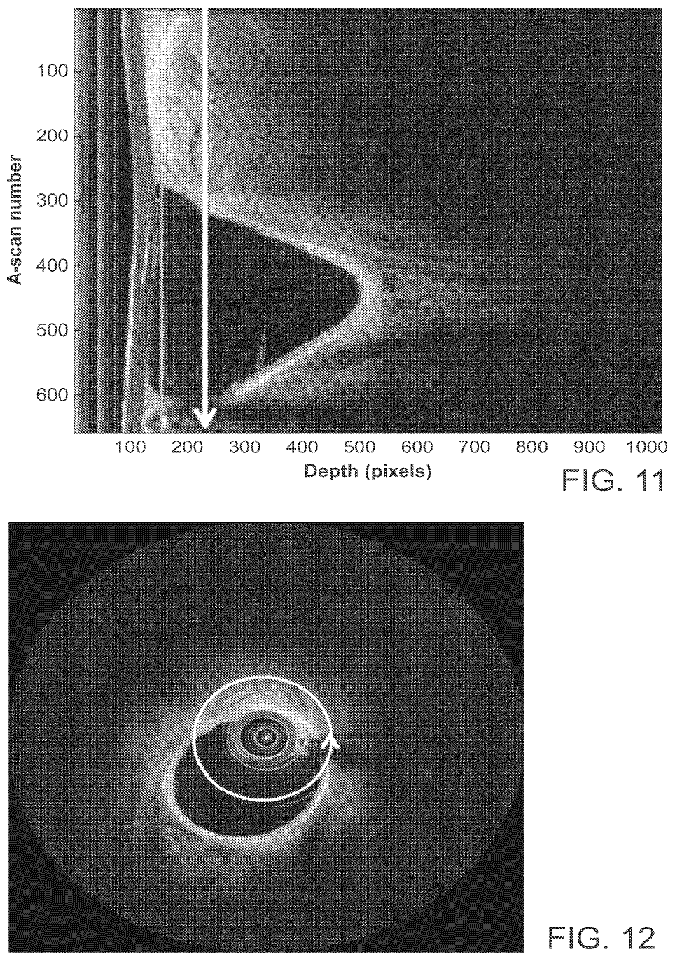

FIG. 11 gives a reproduction of a B-scan collected using an OCT system. Each horizontal row of pixels corresponds to one A-scan, with the first A-scan (e.g., A₁₁) being displayed across the top of the image. The horizontal axis labeled “Depth” represents a radial distance from imaging catheter 826. Noting—as shown in FIG. 9—that each A-scan line is progressively displaced from an adjacent A-scan in an angular direction around an axis 117 of catheter 826 (while also being displaced in a translational direction along axis 117), one set of A-scans associated with a 360° displacement around axis 117 can be collected into a view that depicts a slice of vessel 101 perpendicular to axis 117. This view is referred to as a tomographic view.

FIG. 12 shows a tomographic view based on the B-scan of FIG. 10. A tomographic view comprises a set of A-scans that defines one circumference around vessel 101. An arrow pointing straight down in FIG. 11 corresponds to the circular arrow in FIG. 12 and aids in visualization of the three-dimensional nature of the data.

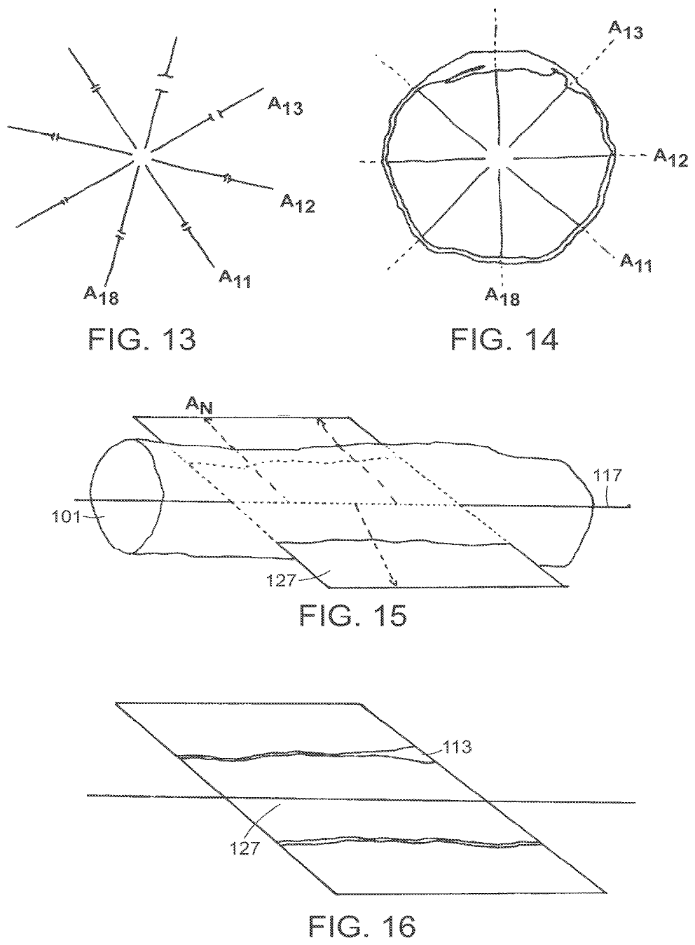

FIG. 13 provides a cartoon illustration of a set of A-scans A₁₁, A₁₂, . . . , A₁₈ used to compose a tomographic view. These A-scan lines are shown as would be seen looking down axis 117 (i.e., longitudinal distance between them is not shown). While eight A-scan lines are here illustrated in cartoon format in FIG. 13, typical OCT applications can include between 300 and 1,000 A-scan lines to create a B scan (e.g., about 660) or a tomographic view.

FIG. 14 provides a cartoon illustration of the tomographic view associated with the A-scans of FIG. 13. Reflections detected along each A-scan line are associated with features within the imaged tissue. Reflected light from each A-scan is combined with corresponding light that was split and sent through reference path 915 and VDL 925 and interference between these two light paths as they are recombined indicates features in the tissue. Where a tomographic view such as is depicted in FIG. 14 generally represents an image as a planar view across a vessel (i.e., normal to axis 117), an image can also be represented as a planar view along a vessel (i.e., axis 117 lies in the plane of the view).

FIG. 15 shows a longitudinal plane 127 through a vessel 101 including several A scans. Such a planar image along a vessel is sometimes referred to as an in-line digital view or image longitudinal display (ILD). As shown in FIG. 15, plane 127 generally comprises data associated with a subset of the A scans. The data of the A scan lines is processed according to systems and methods of the inventions to generate images of the tissue. By processing the data appropriately (e.g., by fast Fourier transformation), a two-dimensional image can be prepared from the three dimensional data set. Systems and methods of the invention provide one or more of a tomographic view, ILD, or both.

FIG. 16 is a perspective view of an idealized plane shown including an exemplary ILD in the same perspective as the longitudinal plane shown in FIG. 15. Where an OCT system captures three-dimensional image data, host workstation 433 may store the three dimensional image data in a tangible, non-transitory memory and provides a display that includes a tomographic view (e.g., FIG. 14), an ILD (e.g., FIG. 16), or both (e.g., on a screen or computer monitor). In some embodiments, a tomographic view and an ILD are displayed together, providing information that operators can intuitively visualize as representing a three-dimensional structure.

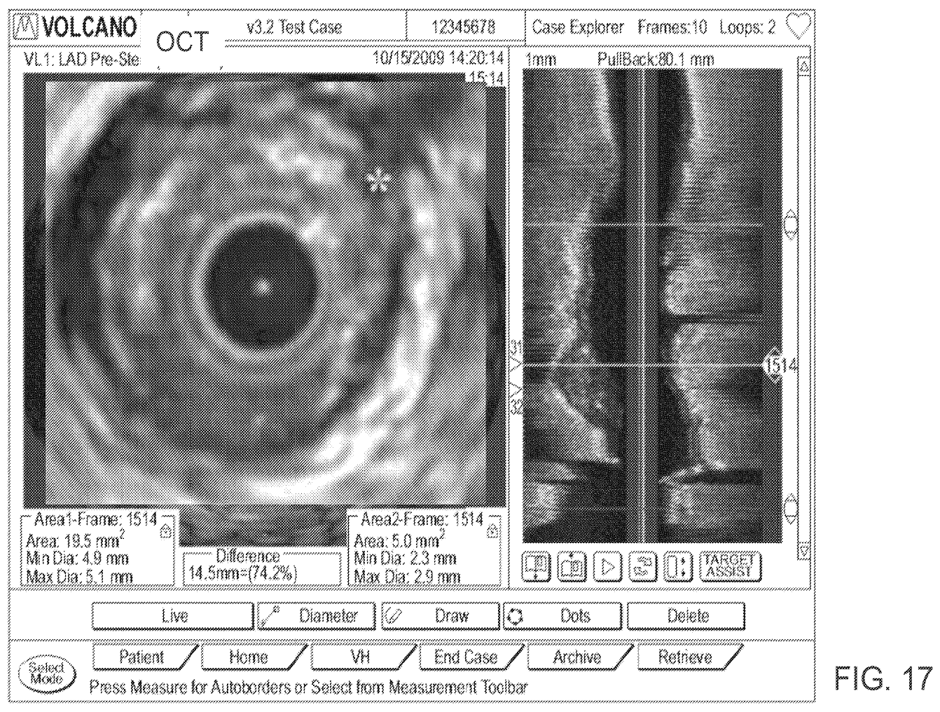

FIG. 17 is a reproduction of a display of an OCT system including a tomographic view on the left and an ILD on the right. As shown in FIG. 17, a tomographic view may include ring-like elements near the center and the ILD may include corresponding sets of vertical line-like elements. One ring in the tomographic view may correspond to one pair of lines in the ILD. These elements within the displays are often, in-fact, images of part of the imaging system itself. In some embodiments, a ring in a tomographic view and lines in an ILD represent a surface of catheter 826 such as, for example, an outer surface of a catheter sheath. The portions of the images extending away from those elements are the images of the patient's tissue.

In some embodiments, an OCT system is operated with interchangeable, replaceable, or single-use catheters. Each catheter 826 may provide a different length to sample path 913. For example, catheters may be used that are designed to be of different lengths, like-manufactured catheters may be subject to imperfect manufacturing tolerances, or catheters may stretch during use. However, to provide a calibrated or scaled image, the z-offset must be known (for post-imaging processing) or set to zero. A z-offset can be known directly (e.g., numerically) or can be known by reviewing an image and determining an apparent difference in an actual location of an element within the image and an expected location of the element within the image.

In some embodiments, the z-offset is calibrated by inspecting an image being captured while they system is running in live mode, and adjusting the actual length of reference path 915 to match the length of sample path 913.

VDL 925 on reference path 915 uses an adjustable fiber coil to match the length of reference path 915 to the length of sample path 913. The length of reference path 915 is adjusted by a stepper motor translating a mirror on a translation stage under the control of firmware or software. The free-space optical beam on the inside of the VDL 925 experiences more delay as the mirror moves away from the fixed input/output fiber. As VDL 925 is adjusted, a length of reference path 915 is known (based, for example, on manufactured specifications of the system).

In some embodiments, the known length of reference path 915 is used to display a calibration mark on a display. If the calibration mark is displayed at a position corresponding to a distal point on reference path 915, and if sample path 913 is the same length as reference path 915 (e.g., when z-offset is zero), it may be expected that a ring in a tomographic view that represents an outer surface of a catheter sheath will lie along the calibration mark.

When a display includes a calibration mark and a ring-like element representing an outer surface of the catheter sheath separated from one another, an operator has a visual indication that the display is not calibrated.

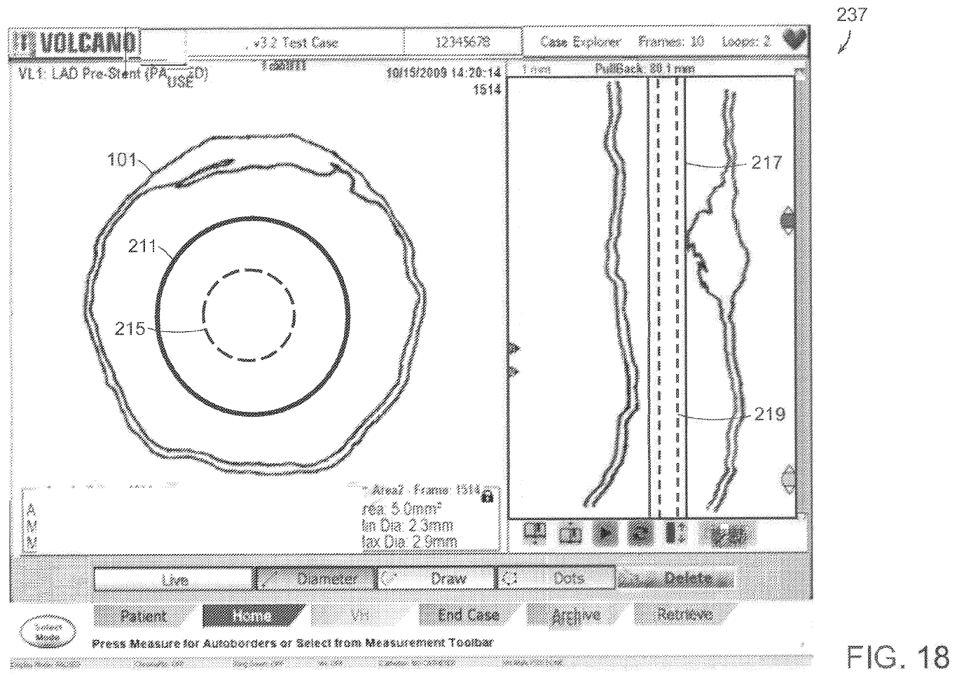

FIG. 18 is a cartoon illustration of a display 237 including an image of the vessel shown in FIGS. 7A and 7B, as rendered by a system of the invention. The images included in display 237 in FIG. 18 are rendered in a simplified style of the purposes of case of understanding. A system of the invention may render a display as shown in FIG. 17, or in any style known in the art (e.g., with or without color).

As shown in FIG. 18, a tomographic view of vessel 101 is depicted alongside an ILD. An outer surface of a catheter sheath appears as a ring 211 in the tomographic view and as lines 217 in the ILD. The tomographic view is depicted as including calibration mark 215, while calibration mark 219 appears in the ILD.

In some embodiments, z-offset calibration involves precisely determining the position of ring 211 (or lines 217) in display 237 so that the system can calculate a z-offset based on a known position of calibration mark 215. Systems of the invention can determine the position of ring 211 or any other calibration element based on user input and an image processing operation. Any suitable user input can be used. In some embodiments discussed below, user input is a “click and drag” operation to move ring 211 to a calibration mark. In certain embodiments, user input is accepted in the form of a single click, a single touch of a touch screen, or some other simple gesture.

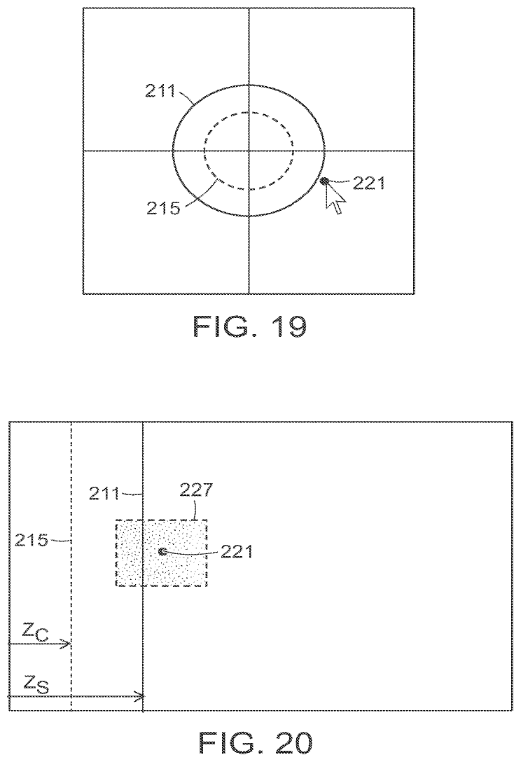

FIG. 19 illustrates, in simplified fashion, a display of an imaging system showing a catheter sheath 211 and calibration mark 215. A user can click on the display near the sheath 211. In some embodiments, the system detects the location of the catheter sheath with no more input from a user than an indication of a single point. A single point can be input by a mouse-click, a touch on a touchscreen, a light pen or light gun, by “driving” a point to a certain position with arrow keys or a joystick, or by any other suitable method known in the art.

The system can additionally use a processor to perform an image processing operation to detect sheath 211. In some embodiments, user input indicates a single point 221 on the screen. The system then defines an area around point 221.

FIG. 20 depicts a defined area 227 around point 221 on a B-scan. Area 227 operates as a search window. The search window area 227 may be a rectangle, circle, ellipse, polygon, or other shape. It may have a predetermined area (e.g., a certain number of pixels). In some embodiments, a size and shape of area 227 is determined by a combination of input device resolution, screen area subtended by a pixel at the particular polar coordinates, current zoom factor, usability studies, or a combination thereof. Usability studies can be performed to establish a statistical model of user repeatability and reproducibility under controlled conditions.

The system searches for the sheath within area 227 by performing a processing operation on the corresponding data. The processing operation can be any suitable search algorithm known in the art.

In some embodiments, a morphological image processing operation is used. Morphological image processing includes operations such as erosion, dilation, opening, and closing, as well as combination thereof. In some embodiments, these operations involve converting the image data to binary data giving each pixel a binary value. With pixels within area 227 converted to binary, each pixel of catheter sheath 211 will be black, and the background pixels will predominantly be white. In erosion, every pixel that is touching background is changed into a background pixel. In dilation, every background pixel that is adjacent to the non-background object pixels is changed into an object pixel. Opening is an erosion followed by a dilation, and closing is a dilation followed by an erosion. Morphological image processing is discussed in Smith, The Scientist and Engineer's Guide to Digital Signal Processing, 1997, California Technical Publishing, San Diego, CA, pp. 436-442.

If sheath 211 is not found within area 227, area 227 can be increased and the increased area can be searched. This strategy can exploit the statistical properties of signal-to-noise ratio (SNR) by which the ability to detect an object is proportional to the square root of its area. See Smith, Ibid., pp. 432-436.

With continued reference to FIG. 20, once a portion of catheter sheath 211 is detected within area 227, the search can then be extended “upwards” and “downwards” into adjacent A-scan lines in the B-scan until the entire catheter sheath 211 is detected by the processor and its location is determined with precision. In some embodiments, image processing operations incorporate algorithms with pre-set or user-set parameters that optimize results and continuity of results. For example, if a line appears that is not contiguous across an entire 100% of the image (e.g., the entire extent of the B-scan or a full circle in a tomographic view), an accept or reject parameter can be established based on a percent contiguous factor. In some embodiments, lines that are contiguous across less than 75% (or 50% or 90%, depending on applications) are rejected while others are accepted.

While described above as detecting a reference item (e.g., catheter sheath 211) by receiving user input followed by using a processor to detect a location of the sheath, the steps can be performed in other orders. For example, the system can apply morphological processing operations to an entire image and detect every element, or every element that satisfies a certain quality criterion. Then the system can receive user input that indicates a point within an image and the user can then choose the pre-detected element that is closest to that point within the image. Similarly, the steps can be performed simultaneously.

Using the methodologies herein, systems of the invention can detect an element within an image of an imaging system, such as an OCT system, with great precision, based on human input that need not be precise and computer processing that need not on its own be accurate. Based on this detection, an actual location of a catheter sheath is determined and thus a precise z-coordinate Zₛ for the catheter sheath (e.g., within a B-scan) is known. Where an expected z-coordinate Z_{c} for the catheter sheath is known, based on information provided extrinsically, the z-offset, and thus a calibration value, can be determined. For example, in FIG. 20, Zₛ is depicted as lying to the right of Z_{c}, thereby showing a non-zero z-offset. The calibration value is then used to provide a calibrated image, or an image at a known scale.

In some embodiments, the system calculates or uses the mean, median, or root-mean-squared distance of the sheath from the calibration mark to compute the calibration value. This may be advantageous in the event of interfering speckle noise, rough or acylindrical sheaths, non-uniform catheter rotation (NURD), angular displacement of a transducer within the sheath, off-center positioning of the transducer within the sheath, or a combination thereof. In certain embodiments, only a subset of the detected points are used, for example, for efficiency or performance optimization.

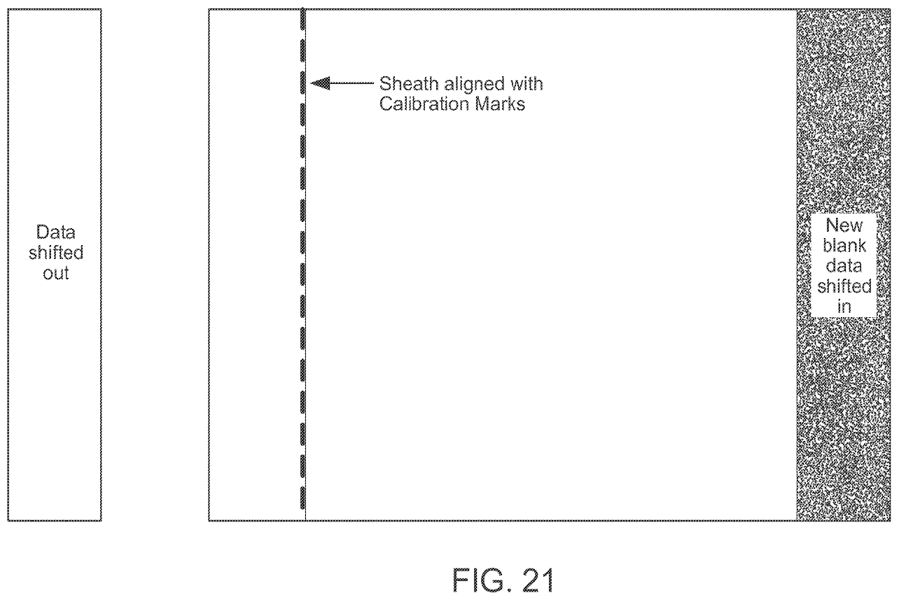

FIG. 21 shows a calibrated image, here, a B-scan. The image is depicted having the catheter sheath aligned with the calibration mark. Bars on the left and right side of FIG. 21 show that some data may be shifted out and some blank space introduced by the calibration. In an alternative embodiment, the image can be stretched or compressed, or a combination of stretching and shifting may be performed, depending on preferences, purposes, or functions of a system.

It will be appreciated that the foregoing description is applicable in live mode or review mode. If the imaging system is operating in live mode, capturing an image of tissue, the calibration can be put into effect either by changing the length of reference path 915 so that z-offset is zero or by transforming the dataset or on-screen image. The length of reference path 915 can be changed through the operation of the motor in the VDL. The distance Z_{c}-Zₛ is converted into millimeters and the a command is sent to move the VDL to a new position.

If the dataset is to be transformed, either in live mode or while the system is operating in review mode, the system is digitally shifted, stretched, or a combination thereof.

In another aspect, the invention provides a method for calibrating an imaging system based on receipt of user input that indicates a “motion”, such as a click-and-drag operation on a computer screen.

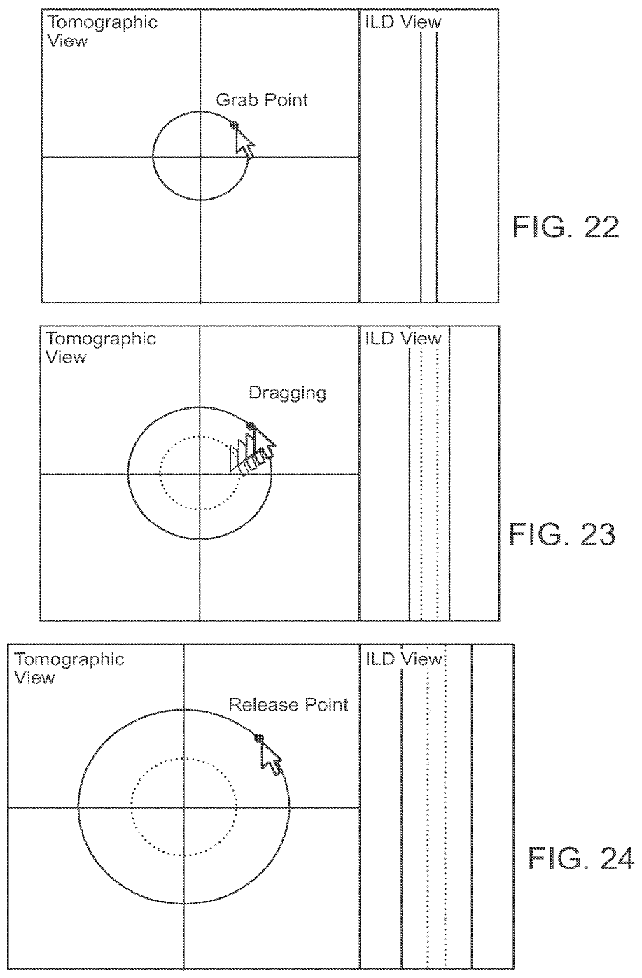

FIGS. 22 and 23 illustrate receiving user input indicating a motion through a mouse dragging operation. User input could also be a drag on a touchscreen or other input (arrow keys, pointer, trackball, etc.) As depicted in FIGS. 22-23, a user clicks on a reference item (e.g., sheath 211) with a mouse and drags it to a new position, for example, onto a calibration mark or other position on the display. The system (e.g., using a processor) can then calculate a calibration value based on indicated motion of the reference item.

This method allows a user to manually calibrate or apply any offset using a drag-and-drop operation on the tomographic view or on the ILD. While dragging, the distance between the grab point and current point represented by the tip of the mouse pointer (or analogous finger-touch point in touchscreens) may be continuously calculated. In live mode, the image may be shifted digitally or by moving the VDL and in review mode the image is transformed digitally, as discussed above.

FIG. 24 shows releasing a click-and-drag motion. In some embodiments, the image is shifted (digitally or by moving the VDL) simultaneously with the user's drag motion. In certain embodiments, the system begins the shift after the user completes the drag input motion. (Note that in FIGS. 23 and 24 a dotted line is shown to represent the original location of the catheter sheath, and the dotted line is not meant to represent a calibration mark. A calibration mark is optional.)

While discussed above using a surface of a catheter sheath as a reference item which is used as a basis for calibration, other reference items are suitable. For example, any item that can be depicted such that its expected location and actual location can be compared in a display of an imaging system may be used. In some embodiments, a fiducial marker or calibration bar is introduced into the imaging target having a known dimension (e.g., 1 nm, 1 mm, 1 cm). The system operates to display a scale or a grid based on an expected appearance of the known dimension. The user then gives input indicating a point in the display near the reference item and the system also detects a location of the reference item in an area around the indicated point. Based on the expected and actual locations or dimensions of the reference item, a calibration value is calculated and a calibrated image is provided. User input, displays, and methods of receiving user input and performing calculations may be provided by one or more computers.

In certain embodiments, display 237 is rendered within a computer operating system environment, such as Windows, Mac OS, or Linux or within a display or GUI of a specialized system. Display 237 can include any standard controls associated with a display (e.g., within a windowing environment) including minimize and close buttons, scroll bars, menus, and window resizing controls. Elements of display 237 can be provided by an operating system, windows environment, application programing interface (API), web browser, program, or combination thereof (for example, in some embodiments a computer includes an operating system in which an independent program such as a web browser runs and the independent program supplies one or more of an API to render elements of a GUI). Display 237 can further include any controls or information related to viewing images (e.g., zoom, color controls, brightness/contrast) or handling files comprising three-dimensional image data (e.g., open, save, close, select, cut, delete, etc.). Further, display 237 can include controls (e.g., buttons, sliders, tabs, switches) related to operating a three dimensional image capture system (e.g., go, stop, pause, power up, power down).

In certain embodiments, display 237 includes controls related to three dimensional imaging systems that are operable with different imaging modalities. For example, display 237 may include start, stop, zoom, save, etc., buttons, and be rendered by a computer program that interoperates with OCT or IVUS modalities. Thus display 237 can display an image derived from a three-dimensional data set with or without regard to the imaging mode of the system.

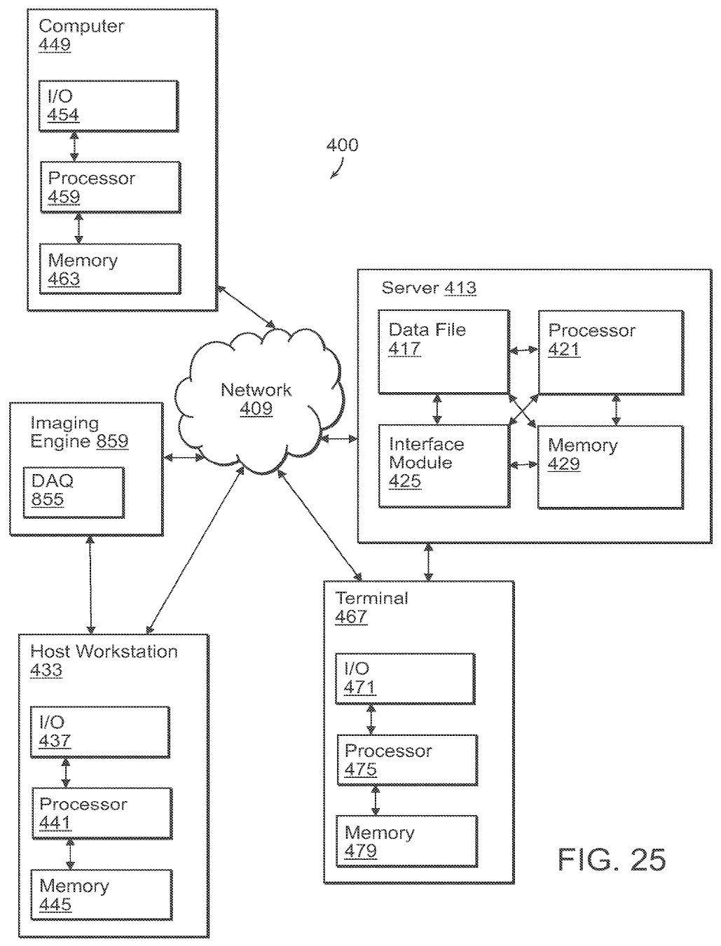

FIG. 25 diagrams an exemplary system 400. As shown in FIG. 25, imaging engine 859 communicates with host workstation 433 as well as optionally server 413 over network 409. In some embodiments, an operator uses host workstation 433, computer 449, or terminal 467 to control system 400 or to receive images. An image may be displayed using an I/O 454, 437, or 471, which may include a monitor. Any I/O may include a monitor, keyboard, mouse or touchscreen to communicate with any of processor 421, 459, 441, or 475, for example, to cause data to be stored in any tangible, nontransitory memory 463, 445, 479, or 429. Server 413 generally includes an interface module 425 to communicate over network 409 or write data to data file 417. Input from a user is received by a processor in an electronic device such as, for example, host workstation 433, server 413, or computer 449. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections). In certain embodiments, host workstation 433 and imaging engine 855 are included in a bedside console unit to operate system 400.

A computer generally includes a processor for executing instructions and one or more memory devices for storing instructions, data, or both. Processors suitable for the execution of methods and operations described herein include, by way of example, both general and special purpose microprocessors (e.g., an Intel chip, an AMD chip, an FPGA). Generally, a processor will receive instructions or data from read-only memory, random access memory, or both. Generally, a computer will also include, or be operatively coupled, one or more mass storage devices for storing data that represent target such as bodily tissue. Any suitable computer-readable storage device may be used such as, for example, solid-state, magnetic, magneto-optical disks, or optical disks. Information carriers suitable for embodying computer program instructions and data include all forms of non-volatile memory, particularly tangible, non-transitory memory including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. (canceled)

2. A system, comprising:
an intravascular imaging catheter configured to be positioned within a vessel of a patient; and
a processor configured for communication with the intravascular imaging catheter, wherein the processor is configured to:
receive, from the intravascular imaging catheter, a first intravascular image comprising the vessel and a reference item;
provide, to a display in communication with the processor, a first output comprising:
the first intravascular image comprising the vessel and the reference item;
a calibration mark on the first intravascular image, wherein the calibration mark is not aligned with the reference item in the first output;
determine a calibration value based on a difference between a location of the calibration mark and a location of the reference item;
transform, based on the calibration value, the first intravascular image into a second intravascular image comprising the vessel and the reference item; and
provide, to the display, a second output without conducting a new scan, wherein the second output comprises:
the second intravascular image comprising the vessel and the reference item; and
the calibration mark on the second intravascular image, wherein the calibration mark is aligned with the reference item in the second output.

3. The system of claim 2, wherein, to transform the first intravascular image into the second intravascular image, the processor is configured to provide relative movement between the location of the calibration mark and the location of the reference item.

4. The system of claim 3, wherein the relative movement is configured to cause the calibration mark to become aligned with the reference item in the second output.

5. The system of claim 2, wherein, to transform the first intravascular image into the second intravascular image, the processor is configured to perform at least one of shifting, stretching, or compressing of the first intravascular image.

6. The system of claim 5, wherein shifting the first intravascular image comprises at least one of:
removal of image data on a first side of the first intravascular image; or
addition of blank space on an opposite, second side of the first intravascular image.

7. The system of claim 2, wherein the reference item comprises a structure of the intravascular imaging catheter disposed within the vessel.

8. The system of claim 7, wherein the reference item comprises a catheter sheath.

9. The system of claim 2, wherein the calibration value comprises a z-offset associated with an interferometric device.

10. The system of claim 2,
wherein the system is an optical coherence tomography system comprising a reference path and a sample path, and
wherein the processor is configured to adjust a length of the reference path based on the calibration value.

11. The system of claim 10,
wherein the reference path comprises a variable delay line, and
wherein, to adjust the length of the reference path, the processor is configured to operate a motor of the variable delay line.
